(19) United States
(12) Patent Application Publication
(10) Pub. No.: US 2005/0154317 A1
(43) Pub. Date: Jul. 14, 2005

(54) Apparatus and method for detecting an acupoint or other site of interest

(76) Inventors: Sang-hoon Shin, Seongnam-si (KR); Woo-young Jang, Seoul (KR)

Correspondence Address: LEE, STERBA & MORSE, P.C., SUITE 2000, 1101 WILSON BOULEVARD, ARLINGTON, VA, 22209, US

(21) Appl. No.: 11/033,554

(22) Filed: Jan. 12, 2005

(30) Foreign Application Priority Data
Jan. 12, 2004 (KR) 2004-0002032

(51) Int. Cl. A61B 6/00

(52) U.S. Cl. 600/473; 600/9

(57) ABSTRACT

In an apparatus for detecting an acupoint using an intensity of biophotons emitted from a living system in response to magnetic field stimuli, and a method for detecting an acupoint, the apparatus includes a magnetic field application unit for applying a magnetic field to a predetermined site of the living system, a biophoton measurement unit for measuring the intensity of the biophotons emitted from the predetermined site of the living system, and an acupoint determination unit for determining whether the predetermined site is an acupoint based on the intensity of the biophotons measured by the biophoton measurement unit.

### BACKGROUND OF THE INVENTION

1. Field of the Invention

The present invention relates to an apparatus and method for detecting an acupoint or other site of interest in a living system. More particularly, the present invention relates to an apparatus and method for easily and precisely detecting the locations of acupoints using a difference in biophoton emission characteristics between acupoints and non-acupoints in response to magnetic field stimuli.

2. Description of the Related Art

Research into biophotons, which are photons emitted from biological systems, began in the early 1920's by Russian scientists that had addressed a question about transfer and modification of information about the sizes and shapes of various different organs by biological tissues. This question is arguably one of the most important issues that must be solved in the biological science field. The Russian scientists asserted that light participates in stimulating cell division through experiments based on onion roots.

In spite of being confirmed by other scientists, after several years, this experimental result was long forgotten due to the absence of an appropriate photon measurement apparatus and the rapid growth of the biochemical field. Even with the rapid development of photomultiplier tubes (PMTs), studies of biological emission in a visible light region, which could not be elucidated by thermal radiation, were still performed by only a few groups, including Inaba (Japan), Boveris (America), and Quichenden (Australia).

In studies of living biological systems, it is very important to detect acupoints to diagnose conditions or diseases of human bodies or efficiently carry out acupressure to facilitate the flow of vital energy or Qi. Therefore, there has been an increasing need to develop an apparatus for detecting acupoints for use in diagnosis and treatments in Chinese medicine, and thus, various studies thereof have been done.

FIG. 1 is a block diagram of a conventional apparatus for detecting acupoints.

Referring to FIG. 1, an apparatus for detecting acupoints includes an optical source unit 101 for emitting light having a predetermined wavelength, an electrical measurement unit 102 for applying a predetermined electrical signal to a test subject 110 and for measuring a transmitted electrical signal to generate an electrical conductivity signal of the test subject 110, a power control unit 104 for applying and controlling a voltage to the optical source unit 101 and the electrical measurement unit 102, an optical receiving unit 103 for receiving light reflected from or transmitted through the test subject 110 and for converting the light to an electrical signal to generate a reflective light signal, a signal processing unit 105 for receiving the light signal from the optical receiving unit 103 and the electrical conductivity signal from the electrical measurement unit 102, and a user supply unit 106 for receiving and analyzing measured data from the signal processing unit 105. The apparatus further includes a power supply unit 109 for supplying power to the signal processing unit 105.

In this conventional acupoint detection apparatus, the signal processing unit 105 receives the reflective light signal from the optical receiving unit 103 and the electrical conductivity signal of the test subject 110 from the electrical measurement unit 102. These signals are converted into digital signals in the signal processing unit 105 and then fed to the user supply unit 106. An incident light fiber 107 transmits an optical signal from the optical source unit 101 to the test subject 110 and an output light fiber 108 transmits light from the test subject 110 to the optical receiving unit 103.

The user supply unit 106 may generate a control signal for controlling the operation of the signal processing unit 105, if necessary. In addition, the user supply unit 106 includes a storage device, and, thus, serves to store the reflective light signal and the electrical conductivity signal. One of output terminals of the user supply unit 106 is connected to an input terminal of the power control unit 104. This completes a feedback system.

However, the above-described conventional acupoint detection apparatus is locally used, and, thus, cannot provide a correlation between acupoints. In addition, a problem may arise in that a weak electrical signal, electrical resistance, and impedance to be measured in biological living systems can vary depending on environmental parameters, such as humidity and temperature, of target sites of the living systems.

### SUMMARY OF THE INVENTION

The present invention is therefore directed to an apparatus and method for detecting acupoints or other sites of interest in a living system, which substantially overcome one or more of the problems due to the limitations and disadvantages of the related art.

It is a feature of an embodiment of the present invention to provide an apparatus and method for detecting acupoints using characteristics of biophotons, which are substantially independent of environmental parameters.

It is another feature of an embodiment of the present invention to provide an apparatus and method for detecting acupoints that are capable of improved detection of acupoints and identification of new acupoints.

It is still another feature of an embodiment of the present invention to provide an apparatus for detecting a site of interest in a living system using an intensity of biophotons emitted from the living system in response to magnetic field stimuli.

At least one of the above features and other advantages may be provided by an apparatus for detecting an acupoint using an intensity of biophotons emitted from a living system in response to magnetic field stimuli including a magnetic field application unit for applying a magnetic field to a predetermined site of the living system, a biophoton measurement unit for measuring the intensity of the biophotons emitted from the predetermined site of the living system, and an acupoint determination unit for determining whether the predetermined site is an acupoint based on the intensity of the biophotons measured by the biophoton measurement unit.

The magnetic field application unit may be operable to apply an ultra-low frequency magnetic field of about 60 Hz or less by adjusting an alternating or direct current.

The biophoton measurement unit may be a photomultiplier tube.

The photomultiplier tube may have an effective diameter of about one centimeter or less.

The biophoton measurement unit may be operable to measure a spatial distribution of emitted biophotons and may be formed as a structure selected from the group consisting of two or more photomultipliers, which operate in close proximity, a multi-channel photomultiplier (MCP) tube, or a charge-coupled device (CCD).

The living body may be a human body, and the acupoint may be one selected from the group consisting of Quze and Neiguan.

At least one of the above features and other advantages may be provided by an apparatus for detecting a site of interest using an intensity of biophotons emitted from a living system in response to magnetic field stimuli, including a magnetic field application unit for applying a magnetic field to a predetermined site of the living system, a biophoton measurement unit for measuring the intensity of the biophotons emitted from the predetermined site of the living system, and a determination unit for determining whether the predetermined site is the site of interest based on the intensity of the biophotons measured in the biophoton measurement unit.

The site of interest may be a tumor. The living system may be a small animal.

In either embodiment, the magnetic field application unit may be operable to apply a magnetic field of about 10 to 1,000 Gauss using a permanent magnet.

In either embodiment, the predetermined site of the living system, which is stimulated by the magnetic field, may have a diameter of about one centimeter or less.

In either embodiment, the photomultiplier tube may be operable to measure an intensity of biophotons having a wavelength of about 200-700 nm.

At least one of the above features and other advantages may be provided by a method for detecting an acupoint using an intensity of biophotons emitted from a living system in response to magnetic field stimuli including dividing a predetermined site of the living system to be measured into n sections, measuring the intensity of the biophotons emitted from an i-th section, where i is an integer from 1 to n, and determining whether the i-th section is an acupoint by comparing the intensity of the biophotons measured on the i-th section to a predetermined value.

Determining whether the i-th section is an acupoint may include calculating an average Iₐ of the intensity of the biophotons and determining whether the i-th section is an acupoint by comparing the intensity Iᵢ of the biophotons measured at the i-th section and the average Iₐ.

The average Iₐ of the intensity of the biophotons may be calculated by the following equation:

$${I}_{a} = \frac{{\sum }_{i = 1}^{n} {I}_{i}}{n} ,$$

wherein Iₐ is the average of the intensities of the biophotons and n is the number of the divided sections of the predetermined site of the living system to be measured.

Determining whether the i-th section is an acupoint by comparing the intensity Iᵢ of the biophotons measured at the i-th section and the average Iₐ may be performed using the following equation:
|Iₐ−Iᵢ|>Iₜₕ,
wherein Iₐ is the average of the intensities of biophotons measured, Iᵢ is the intensity of biophotons measured in the i-th section, and Iₜₕ is a predetermined value.

Iₜₕ may be an intensity difference when a difference between the intensities of biophotons emitted from an acupoint and a non-acupoint, as measured for about one minute, is about fifty.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1 is a functional block diagram of a conventional apparatus for detecting acupoints;

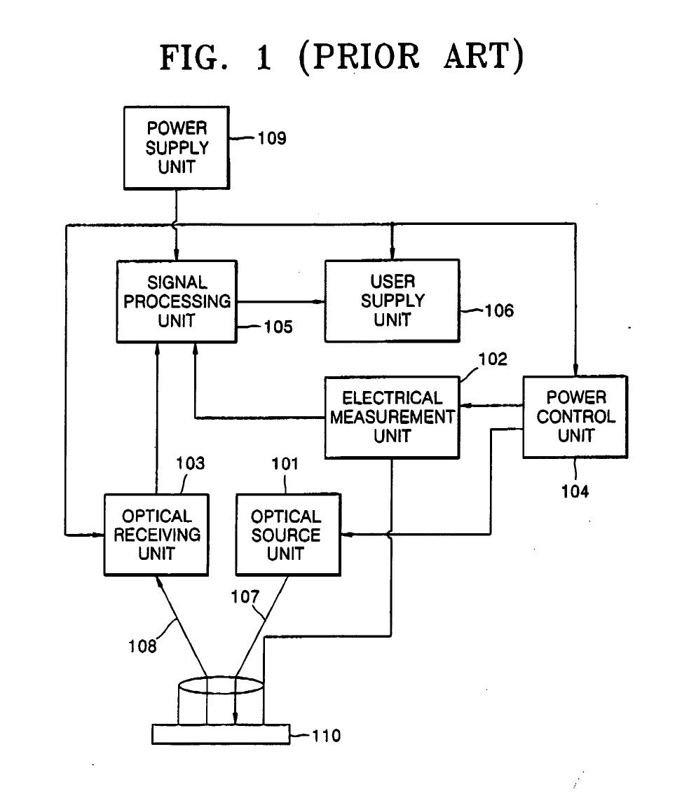

FIG. 2 is a schematic block diagram of an apparatus for detecting acupoints using a change in an intensity of biophotons emitted in response to magnetic field stimuli according to an embodiment of the present invention;

FIG. 3 illustrates several acupoints in a human body to be detected according to an embodiment of the present invention;

FIG. 4 is a graph illustrating experimental data according to an embodiment of the present invention; and

FIG. 5 is a flowchart of a method for detecting acupoints using a change in an intensity of biophotons emitted in response to magnetic field stimuli according to an embodiment of the present invention.

### DETAILED DESCRIPTION OF THE INVENTION

Korean Patent Application No.10-2004-0002032, filed on Jan. 12, 2004, in the Korean Intellectual Property Office, and entitled: “Apparatus and Method for Detecting Acupoints,” is incorporated by reference herein in its entirety.

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. In the drawings, the thicknesses of layers and regions are exaggerated for clarity. Like reference numerals and characters indicate like elements throughout.

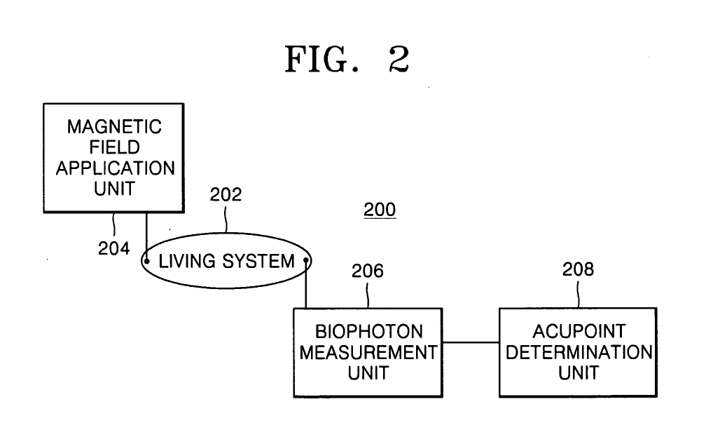

FIG. 2 is a schematic block diagram of an apparatus for detecting acupoints using a change in an intensity of biophotons emitted in response to magnetic field stimuli according to an embodiment of the present invention.

Referring to FIG. 2, an apparatus 200 for detecting acupoints using a change in the intensity of biophotons emitted in response to magnetic field stimuli includes a magnetic field application unit 204 for applying a magnetic field to a predetermined site of a living system 202, which is a test subject, a biophoton measurement unit 206, and a determination unit 208, e.g., an acupoint determination unit, for determining whether the predetermined site is an acupoint based on data output from the biophoton measurement unit 206.

In operation, the magnetic field application unit 204 applies a magnetic field of about 10 to 1,000 Gauss using a permanent magnet.

Alternatively, the magnetic field application unit 204 may apply an ultra-low frequency magnetic field of about 60 Hz or less by adjusting an alternating or direct current.

The predetermined site of the living system 202 may have a diameter of about one centimeter and the magnetic field application unit 204 may be designed so that the predetermined site of the living system 202 having a diameter of about one centimeter or less is stimulated by the magnetic field.

The biophoton measurement unit 206 may preferably be a shielding system, including a photomultiplier tube, which operates according to the following principle. Electrons are reflected back from a surface of a solid after collision between the electrons and the solid. At the same time, a collision energy is transmitted from the electrons to electrons in the solid, thereby ejecting excited electrons from the solid. This phenomenon is called secondary electron emission. Based on this phenomenon, microscale photoelectrons can be multiplied, and the multiplied signals can be detected.

The photomultiplier tube may include a shutter and a preamplifier. The shutter remains closed until the biophoton measurement is initiated in order to prevent damage to the photomultiplier that may be caused by exposure to common indoor electric light. The photomultiplier tube is able to measure biophotons having the intensity as weak as one-millionth of starlight. To measure biophotons having such a weak intensity, it is preferable to perform the measurement in a dark room completely shielded from outside light.

The present invention has been made based on biophotons being emitted in different patterns according to a condition of a human body. The photomultiplier that can be used herein is generally a device that is operable to measure biophotons emitted from solids. Since one biophoton is amplified about millions of times and then its density is measured, it is preferable to make the photomultiplier as a device that is capable of measuring an ultra-weak light. The photomultiplier can measure the number of incident photons, and thus, may be called a “single photon counter.”

The intensity of the biophotons measured by the photomultiplier may be displayed on a computer/counting board (not shown) via the preamplifier so that the measurement result may be viewed in real time. At this time, the preamplifier converts the intensity of the biophotons measured by the photomultiplier to a voltage and then amplifies the voltage.

The photomultiplier may preferably have an effective diameter of about one centimeter or less and may preferably be operable to measure biophotons having a wavelength of about 200-700 nm.

The biophoton measurement unit 206 may be formed as a structure having two or more photomultipliers, which operate in close proximity, a multi-channel photomultiplier (MCP) tube, or a charge-coupled device (CCD), to measure spatial distribution of biophotons emitted.

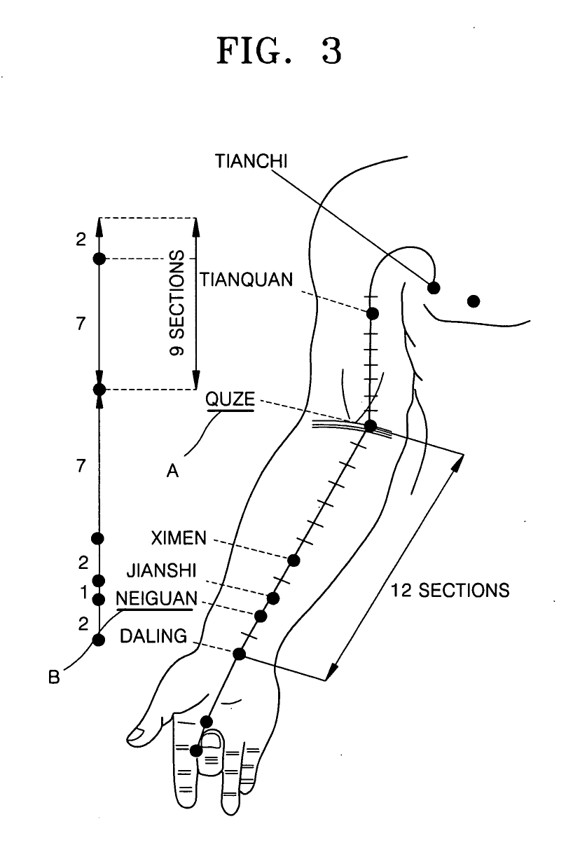

In the acupoint detection apparatus 200, the magnetic field application unit 204 may be designed so that only local specific sites of skin surface of the living system 202, i.e., acupoints, are stimulated. A magnetic field to be applied may be produced by a permanent magnet or an electromagnet and create a waveform periodically changing with time. Exemplary acupoints will be described hereinafter with reference to FIG. 3.

After magnetic stimuli are applied to the living system 202 by the magnetic field application unit 204, the biophoton measurement unit 206 measures light emitted from stimulated local sites of the living system 202, and, at the same time, light emitted from other local sites of the living system 202 other than the stimulated local sites.

A site intended for magnetic field application and a site intended for biophoton measurement may be defined to have a diameter of about one centimeter or less. Accordingly, a distance between the two sites must be at least about two centimeters. However, these values may vary depending on the effective areas of the magnetic field application unit 204 and the biophoton measurement unit 206.

The biophoton measurement unit 206 may further include a computer to collect and analyze data.

In operation, the acupoint determination unit 208 compares the intensity of biophotons emitted from a predetermined site with that from a known acupoint and determines whether the predetermined site is a new acupoint based on a predetermined value. The predetermined value may preferably be a value that corresponds to an intensity difference when a difference between the total number of biophotons emitted from an acupoint and a non-acupoint, as measured for about one minute, is about fifty. The difference value, however, may vary depending on experimental conditions.

FIG. 3 illustrates several exemplary acupoints in a human body to be detected according to an embodiment of the present invention. In FIG. 3, “A” indicates Quze of the pericardium meridian and “B” indicates Neiguan of the pericardium meridian. The Quze and Neiguan are representative acupoints in a human body.

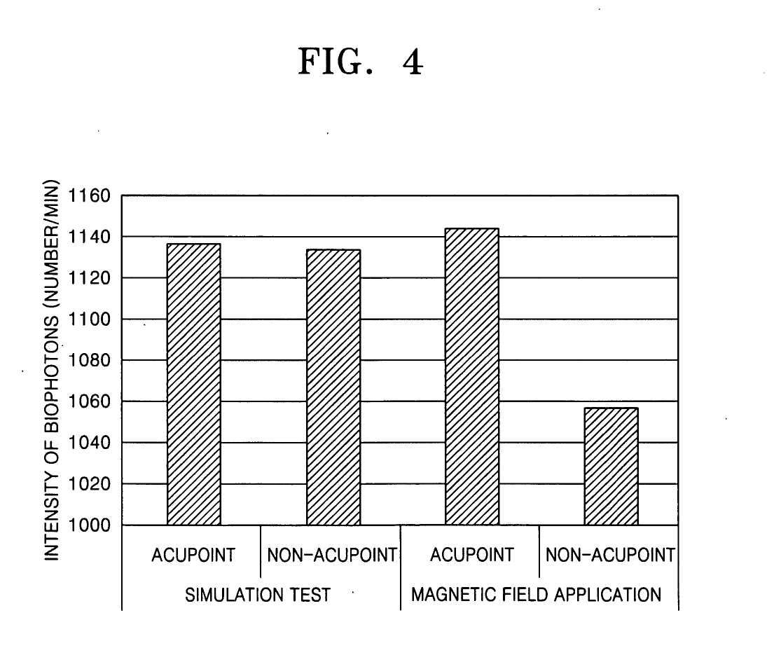

FIG. 4 is a graph that illustrates experimental data in a human body according to an embodiment of the present invention. More specifically, FIG. 4 illustrates experimental data in the two acupoints A and B shown in FIG. 3, i.e., the Quze and the Neiguan, which are experientially well known to be located on a blood vessel running from a point near the elbow to a point near the wrist.

First, while magnetic stimuli are applied to the Quze, which is an acupoint located on the inner flexure of the elbow, the intensity of biophotons emitted from the Neiguan, which is another acupoint located near the wrist, as shown in FIG. 3, is measured.

To show a difference between the intensities of biophotons emitted from an acupoint and a non-acupoint, the intensity of biophotons emitted from a non-acupoint spaced about two centimeters apart from the Neiguan is measured.

As shown in FIG. 4, in connection with simulation tests performed without magnetic field stimuli, no difference appears between the intensities of biophotons emitted from an acupoint and a non-acupoint. When a magnetic field of about 500 Gauss is applied to a human body, however, there is a distinct difference between the intensities of biophotons emitted from the acupoint and the non-acupoint.

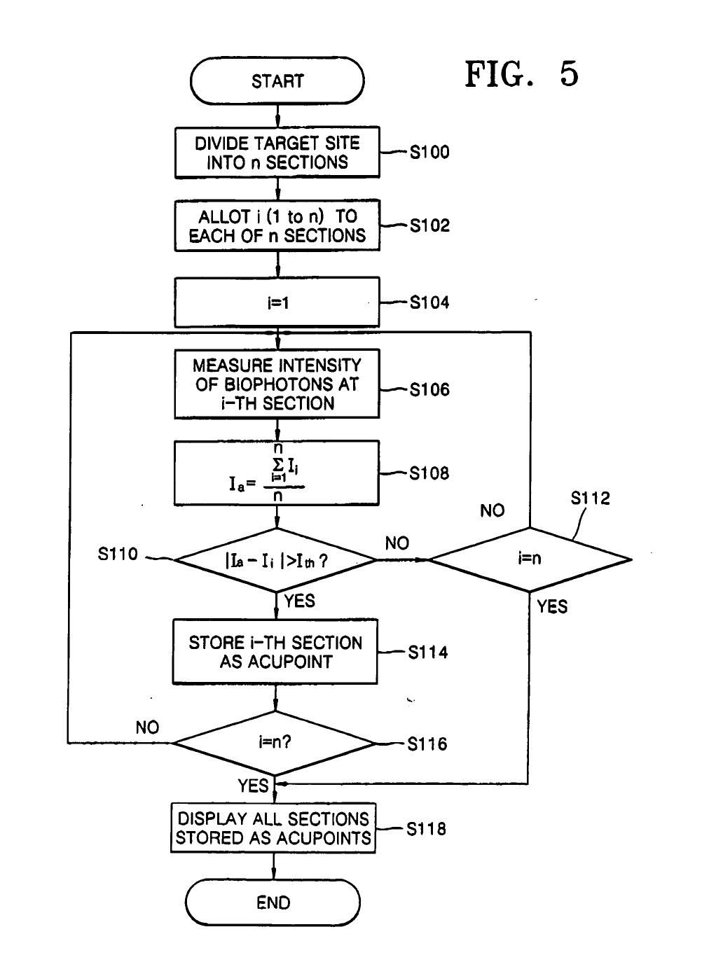

FIG. 5 is a flowchart of a method for detecting an acupoint using a change in an intensity of biophotons emitted in response to magnetic field stimuli according to an embodiment of the present invention. Referring to FIG. 5, a method for detecting an acupoint using a change in the intensity of biophotons emitted in response to magnetic field stimuli includes steps S100 through S118.

Hereinafter, a method for detecting an acupoint using a change in the intensity of biophotons emitted in response to magnetic field stimuli as shown in FIG. 5 will be described with reference to the acupoint detection apparatus 200 shown in FIG. 2.

In step S100, a target site of the living system 202 to be measured is divided into n sections. Although not shown, the target site of the living system 202 may be shielded from surrounding light.

In step S102, a serial number i is allotted to each of the n sections, where i is an integer from 1 to n and n is an integer greater than 1.

In step S104, i is initially set equal to 1. The n sections may preferably be scanned by incrementing i by one or more. Alternatively, a zigzag scan may be applied.

In step S106, an intensity Iᵢ of biophotons emitted from an i-th section is measured.

In step S108, an average Iₐ of the intensities of biophotons emitted using the measured intensities Iᵢ is calculated using Equation 1:

$${I}_{a} = \frac{{\sum }_{i = 1}^{n} {I}_{i}}{n} , \qquad ( 1 )$$

wherein Iₐ is the average of the intensities of biophotons and n is the number of the divided sections of the target site of the living system to be measured.

In step S110, whether the i-th section is an acupoint is determined using Equation 2, based on the intensity Iᵢ of biophotons measured in the i-th section:
|Iₐ−Iᵢ|>Iₜₕ (2)
wherein Iₐ is an average of the intensities of biophotons, Iᵢ is the intensity of biophotons measured in the i-th section, and Iₜₕ is a predetermined value. Iₜₕ may preferably be an intensity difference when a difference between the intensities of biophotons emitted from an acupoint and a non-acupoint, as measured for about one minute, is about fifty.

In step S110, if a determination result is negative, then, in step S112, it is determined whether i=n. If a determination result in step S112 is affirmative, then the method proceeds to step S118. If the determination result in step S112 is negative, then step S106 is repeated. If the determination result in step S110 is affirmative, then the method proceeds to step S114. In step S114, the i-th section is stored as an acupoint.

Next, in step S116, it is determined whether i=n. If the determination result in step S116 is affirmative, then the method proceeds to step S118. If the result is negative, step S106 is repeated.

Finally, after the measurements of the intensities of biophotons on the n sections are completed, in step S118, all sections corresponding to acupoints stored in step S114 are displayed.

Even though the present invention has been illustrated in the context of detecting acupoints, it is understood that the present invention may be used to detect specific sites of interest in a living system other than acupoints, e.g., tumors.

In the method for detecting acupoints using a change in the intensity of biophotons emitted in response to magnetic field stimuli according to an embodiment of the present invention, the average Iₐ is calculated using the intensity Iᵢ of biophotons emitted from the i-th section. However, it is understood that a previously calculated average for the intensities of biophotons emitted from several sites other than acupoints can be used.

While the present invention has been illustrated in view of a human body, considering the importance of animal tests conducted in the medical field, in particular, in the field of Chinese medicine, the present invention can be used to detect specific sites of interest of a small animal, such as a white rat, and measurement of the intensity of biophotons emitted from the specific sites in response to magnetic field stimuli, for the purpose of diagnosis and treatments in Chinese medicine.

As is apparent from the above description of an embodiment of the present invention, acupoints can be detected based on the intensity of biophotons emitted from specific sites of a living system in response to magnetic field stimuli, thereby enabling identification of new acupoints.

The apparatus and method of the present invention can be further used in the detection of sites of interest, such as tumors, of a living system and in studies of a living system based on blood vessels and acupoints in Chinese medicine. Furthermore, the apparatus and method of the present invention can be utilized in medicinal diagnosis.

In addition, since biophotons are substantially independent of environmental parameters, more accurate detection of acupoints is possible.

Exemplary embodiments of the present invention have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

1. An apparatus for detecting a site of interest using an intensity of biophotons emitted from a living system in response to magnetic field stimuli, comprising:
a magnetic field application unit for applying a magnetic field to a predetermined site of the living system;
a biophoton measurement unit for measuring the intensity of the biophotons emitted from the predetermined site of the living system; and
a determination unit for determining whether the predetermined site is a site of interest based on the intensity of the biophotons measured by the biophoton measurement unit.

2. The apparatus as claimed in claim 1, wherein the magnetic field application unit is operable to apply a magnetic field of about 10 to 1,000 Gauss using a permanent magnet.

3. The apparatus as claimed in claim 1, wherein the magnetic field application unit is operable to apply an ultra-low frequency magnetic field of about 60 Hz or less by adjusting an alternating or direct current.

4. The apparatus as claimed in claim 1, wherein the predetermined site of the living system, which is stimulated by the magnetic field, has a diameter of about one centimeter or less.

5. The apparatus as claimed in claim 1, wherein the biophoton measurement unit is a photomultiplier tube.

6. The apparatus as claimed in claim 5, wherein the photomultiplier tube is operable to measure an intensity of biophotons having a wavelength of about 200-700 nm.

7. The apparatus as claimed in claim 6, wherein the photomultiplier tube has an effective diameter of about one centimeter or less.

8. The apparatus as claimed in claim 1, wherein the biophoton measurement unit is operable to measure a spatial distribution of emitted biophotons and is formed as a structure selected from the group consisting of two or more photomultipliers, which operate in close proximity, a multi-channel photomultiplier (MCP) tube, or a charge-coupled device (CCD).

9. The apparatus as claimed in claim 1, wherein the living body is a human body, the site of interest is an acupoint, and the determination unit is an acupoint determination unit.

10. A method for detecting a site of interest using an intensity of biophotons emitted from a living system in response to magnetic field stimuli, comprising:
dividing a predetermined site of the living system to be measured into n sections;
measuring the intensity of the biophotons emitted from an i-th section, where i is an integer from 1 to n; and
determining whether the i-th section is a site of interest by comparing the intensity of the biophotons measured on the i-th section to a predetermined value.

11. The method as claimed in claim 10, wherein determining whether the i-th section is a site of interest, comprises:
calculating an average Iₐ of the intensity of the biophotons; and
determining whether the i-th section is a site of interest by comparing the intensity Iᵢ of the biophotons measured at the i-th section and the average Iₐ.

12. The method as claimed in claim 11, wherein the average Iₐ of the intensity of the biophotons is calculated by the following equation:
$${I}_{a} = \frac{{\sum }_{i = 1}^{n} {I}_{i}}{n} ,$$
wherein Iₐ is the average of the intensities of the biophotons and n is the number of the divided sections of the predetermined site of the living system to be measured.

13. The method as claimed in claim 12, wherein determining whether the i-th section is a site of interest by comparing the intensity Iᵢ of the biophotons measured at the i-th section and the average Iₐ is performed using the following equation:

|Iₐ−Iᵢ|>Iₜₕ,
wherein Iₐ is the average of the intensities of biophotons measured, Iᵢ is the intensity of biophotons measured in the i-th section, and Iₜₕ is a predetermined value.

14. The method as claimed in claim 13, wherein Iₜₕ is an intensity difference when a difference between the intensities of biophotons emitted from a site of interest and a site not of interest, as measured for about one minute, is about fifty.

15-18. (canceled)

19. The apparatus as claimed in claim 1, wherein the site of interest is a tumor.

20. The apparatus as claimed in claim 1, wherein the living system is a small animal.

21. The apparatus as claimed in claim 9, wherein the acupoint is one selected from the group consisting of Quze and Neiguan.

22. The method as claimed in claim 10, wherein the site of interest is an acupoint.
